SEQUENCE LISTING (3 sequences)

SEQ ID NO: 1 - DNA, 25 bp, Artificial Sequence
  Synthetic Construct
aacacuuacacucggaaaugauggg

SEQ ID NO: 2 - DNA, 24 bp, Artificial Sequence
  Synthetic Construct
aacacuuacacucggaaaugaugg

SEQ ID NO: 3 - DNA, 21 bp, Artificial Sequence
  Synthetic Construct
uucuccgaacgugucacgutt

(19) United States
(12) Patent Application Publication
(10) Pub. No.: US 2019/0105395 A1
(43) Pub. Date: Apr. 11, 2019

(54) CYTOCOMPATIBLE HYDROGELS FOR ENCAPSULATION OF CELLS

(71) Applicant: CASE WESTERN RESERVE UNIVERSITY, Cleveland, OH (US)

(72) Inventors: Eben Alsberg, Cleveland, OH (US); Fangze Liu, Cleveland, OH (US); Yuxuan Cheng, Cleveland, OH (US); Katherine A. Coughlin, Cleveland, OH (US)

(21) Appl. No.: 16/153,138

(22) Filed: Oct. 5, 2018

Related U.S. Application Data
(60) Provisional application No. 62/568,612, filed on Oct. 5, 2017.

(51) Int. Cl. A61K 47/34; C12N 15/113; C08J 3/075; A61K 35/28; A61K 9/06; A61L 27/52; A61L 27/54; A61L 27/58; A61L 27/26; A61L 27/18

(52) U.S. Cl. CPC A61K 47/34; C08J 2471/00; C08J 3/075; A61K 35/28; A61K 9/06; A61L 27/52; A61L 27/54; A61L 27/58; A61L 27/26; A61L 27/18; C12N 2310/14; C12N 2310/141; C12N 2320/32; A61L 2430/34; A61L 2400/06; A61L 2300/604; A61L 2300/64; C08J 2363/00; C08J 2371/00; C08J 2463/00; C12N 15/113

(57) ABSTRACT

A cytocompatible hydrogel includes a plurality first polymer macromers linked to a plurality of second polymer macromers different than the first polymer macromers by a plurality of β-hydroxythio-ether linkages, and a plurality of cells encapsulated in the hydrogel.

### RELATED APPLICATION

This application claims priority from U.S. Provisional Application No. 62/568,612, filed Oct. 5, 2017, the subject matter of which is incorporated herein by reference in its entirety.

### GOVERNMENT FUNDING

This invention was made with government support under Grant Nos. AR069564 awarded by The National Institutes of Health. The United States government has certain rights to the invention.

### BACKGROUND

Hydrogel biomaterials are three-dimensional (3D) networks of crosslinked hydrophilic polymers that contain a large fraction of water, and have been widely explored as biomaterial matrices for use in applications, such as therapeutic delivery, wound dressing and tissue engineering. Hydrogel biomaterials play an important role in the field of regenerative medicine and tissue engineering by offering the potential to encapsulate cells with minimal cytotoxicity. Additionally, hydrogel biomaterials possess a highly hydrated, porous structure that may provide the capacity for localized, sustained and/or controlled release of bioactive molecules and facilitate the transport of nutrients and oxygen to incorporated cells and the removal of waste. A wide range of natural and synthetic polymers including alginate, collagen, chitosan, gelatin, polypeptides, and poly(ethylene glycol) (PEG), and a variety of mechanisms, such as self-assembly, ionic crosslinking and covalent crosslinking, have been utilized to fabricate hydrogel biomaterials. Among these chemistries, covalent crosslinking including free radical photopolymerization, Michael-type addition, click reactions, Schiff base reaction, and enzyme-mediated reactions has been extensively used to fabricate hydrogels for tissue engineering.

### SUMMARY

Embodiments described herein relate to cytocompatible hydrogels, methods of forming the hydrogels, and to their use in regenerative medicine, cell-based technologies, drug delivery, and tissue engineering applications. The cytocompatible hydrogels include a plurality first polymer macromers crosslinked with a plurality of second polymer macromers different than the first polymer macromers by a plurality of β-hydroxythio-ether linkages. A plurality of cells and, optionally, bioactive agents can be encapsulated in the hydrogel. The hydrogel upon degradation, can produce substantially non-toxic products. Advantageously, the cytocompatible hydrogel can readily support three dimensional cell culture of encapsulated cells and provide a platform for delivery of bioactive agents to the encapsulated cells or to tissue to which the hydrogel is administered.

In some embodiments, the first polymer macromers are crosslinked with the second polymer macromers by reaction of a plurality of epoxy polymer macromers with a plurality thiolated polymer macromers in a basic aqueous solution. The epoxy polymer macromers can include, for example, epoxy poly(ethylene glycol) macromers, and the thiolated polymer macromers can include, for example, thiolated poly(ethylene glycol) macromers.

In some embodiments, the thiolated poly(ethylene glycol) macromers can be n-arm-poly(ethylene glycol-thiol)macromers, wherein n is 3 or more, and the epoxy poly(ethylene glycol) macromers can be diepoxy poly(ethylene glycol) macromers.

In other embodiments, at least one bioactive agent can be encapsulated in the hydrogel. The bioactive can include, for example, at least one of a peptide, protein, polynucleotide, or small molecule that modulates a function and/or characteristic of at least one of the encapsulated cells. The polynucleotide can include DNA fragments, DNA plasmids, and/or interfering RNA molecules that modulate a function and/or characteristic of at least one of the encapsulated cells. In some embodiments, interfering RNA molecules can include siRNA or miRNA.

In some embodiments, the encapsulated cells can include at least one of progenitor cells, undiffentiated cells, and/or differentiated cells. For example, the cells can include human mesenchymal stem cells.

Other embodiments described herein relate to a method of forming a hydrogel based biomaterial. The method can include reacting a plurality of epoxy polymer macromers with a plurality thiolated polymer macromers in a basic aqueous solution to form a hydrogel. A plurality of cells and optionally, a bioactive agent, can be loaded into the hydrogel during gelation of the hydrogel. The hydrogel so formed is cytocompatible, and, upon degradation, produces substantially non-toxic products.

In some embodiments, the epoxy polymer macromers are epoxy poly(ethylene glycol) macromers, and the thiolated polymer macromers are thiolated poly(ethylene glycol) macromers. For example, the thiolated poly(ethylene glycol) macromers can be n-arm-poly(ethylene glycol-thiol)macromers, wherein n is 3 or more, and the epoxy poly(ethylene glycol) macromers can be diepoxy poly(ethylene glycol) macromers.

In some embodiments, the cells loaded into the hydrogel can include at least one of progenitor cells, undiffentiated cells, and/or differentiated cells. For example, the cells can include human mesenchymal stem cells.

In other embodiments, the bioactive agent loaded into the hydrogel can include, for example, at least one of a peptide, protein, polynucleotide, or small molecule that modulates a function and/or characteristic of at least one of the cells. The polynucleotide can include DNA fragments, DNA plasmids, and/or interfering RNA molecules that modulate a function and/or characteristic of at least one of the cells. In some embodiments, interfering RNA molecules can include siRNA or miRNA.

### BRIEF DESCRIPTION OF THE DRAWINGS

FIGS. 1(A-D) illustrate: chemical structure of (A) PEG-diepoxy (PEG-DE) and (B) 8-arm PEG-thiol (PEG(-SH)₈) macromers. (C) Proton NMR with assigned protons of the macromer mixture at different time points. (D) 1³C NMR with assigned carbons of (1) PEG-DE, (2) PEG(-SH)₈, and their mixture at (3) neutral and (4) basic pH. NMR data shows the appearance of proton (C) and carbon (D) peaks in the newly formed β-hydroxyl thioether groups.

FIGS. 2(A-D) illustrate graphs showing (A) Gelation time determined by tube inversion method. (B) Rheology of combined macromer solutions over time at 25° C. (C, D) Live/dead staining of hMSCs encapsulated in the hydrogels (C) without and (D) with RNA/PEI complexes cultured in growth media.

FIGS. 3(A-E) illustrate a schematic showing preparation of hMSC-encapsulated hydrogels via thiol-epoxy “click” chemistry in the (A) absence or (B) presence of siNog/PEI nanocomplexes for regulating hMSC osteogenic differentiation (C-E).

FIGS. 4(A-C) illustrate plots and images showing biochemical assay for osteogenesis of hMSCs of (A) Donor 1 and (B) Donor 2 showing DNA (1) and Ca (2) per gel volume unit, and DNA normalized Ca amount (3). (C) Histology images of the sectioned gels (Donor 1) stained with ARS for Ca visualization showing consistent results with much more Ca in siNog/OM compare to others condition. * p<0.05; # p<0.05 compared to the same group at D7; & p<0.05 compared to the same group at other time points; +p<0.05 compared to the same group at D14.

FIG. 5 illustrates a schematic thiol-epoxy “click” hydrogel formation with encapsulated hMSCs and siRNA/PEI complexes for tissue regeneration applications.

FIG. 6 illustrates a graph showing gelation time of thiol-epoxy click hydrogels formed with PEG-thiol macromers of different structures determined via the tube inversion method. *′ ^{&} p<0.001 compared to groups that possess the same symbol.

FIGS. 7(A-B) illustrate plots showing swelling ratio of hydrogels, which were prepared from PEG(SH)₈ (10 kDa) and PEG-DE (500 Da): (A) in PBS pH 7.4 (a,b: p<0.05 compared to day 1 (a) and 7 (b) for 7.5% gels; c: p<0.05 compared to day 1 for all gel concentrations; d: p<0.05 compared to day 1 and 7 for all gel concentrations) and (B) in DMEM-HG (a: p<0.05 compared to day 1 for 15% gels; b: p<0.05 compared to day 1 for all gel concentrations; c: p<0.01 compared to day 1 and 7 for all gel concentrations; d: p<0.05 compared to day 14 for 10 and 15% gels).

FIG. 8 illustrates a graph showing the quantification of cell viability in live/dead staining images using Fiji software. No significant difference was found.

FIGS. 9(A-C) illustrate graphs showing (A) DNA content, (B) Ca content, and (C) Ca/DNA of the thiol-epoxy hydrogels with encapsulated hMSCs (Donor 2) with or without siNog cultured in osteogenic media.

FIGS. 10(A-C) illustrate graphs and images showing biochemical and histological evaluation of empty gels (Gel-alone) cultured in both GM and OM compared to gels containing hMSCs (hMSCs-Gel) cultured in GM. (A) DNA and (B) Ca content after 4 weeks of culture. * p<0.05 compared to other groups. (C) Histological photomicrographs samples cultured for 4 weeks, sectioned and stained with ARS.

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the present invention pertains. Commonly understood definitions of molecular biology terms can be found in, for example, Rieger et al., Glossary of Genetics: Classical and Molecular, 5th Ed., Springer-Verlag: New York, 1991, and Lewin, Genes V, Oxford University Press: New York, 1994. The definitions provided herein are to facilitate understanding of certain terms used frequently herein and are not meant to limit the scope of the present invention.

The term “bioactive agent” can refer to any agent capable of promoting tissue growth, inhibition, formation, destruction, and/or targeting a specific disease state. Examples of bioactive agents can include, but are not limited to, chemotactic agents, various proteins (e.g., short term peptides, bone morphogenic proteins, collagen, glycoproteins, and lipoprotein), cell attachment mediators, biologically active ligands, integrin binding sequence, various growth and/or differentiation agents and fragments thereof (e.g., epidermal growth factor (EGF), hepatocyte growth factor (HGF), vascular endothelial growth factors (VEGF), fibroblast growth factors (e.g., bFGF), platelet derived growth factors (PDGF), insulin-like growth factor (e.g., IGF-I, IGF-II) and transforming growth factors (e.g., TGF-β I-III), parathyroid hormone, parathyroid hormone related peptide, bone morphogenic proteins (e.g., BMP-2, BMP-4, BMP-6, BMP-7, BMP-12, BMP-13, BMP-14), transcription factors, such as sonic hedgehog, growth differentiation factors (e.g., GDF5, GDF6, GDF8), recombinant human growth factors (e.g., MP52 and the MP-52 variant rhGDF-5), cartilage-derived morphogenic proteins (CDMP-1, CDMP-2, CDMP-3), small molecules that affect the upregulation of specific growth factors, DNA fragments, DNA plasmids, MMPs, TIMPs, interfering RNA molecules, such as siRNAs, oligonucleotides, proteoglycans, glycoproteins, glycosaminoglycans, and DNA encoding for shRNA.

The terms “biodegradable” and “bioresorbable” may be used interchangeably and refer to the ability of a material (e.g., a natural or synthetic polymer or macromer) to be fully resorbed in vivo. “Full” can mean that no significant extracellular fragments remain. The resorption process can involve elimination of the original implant material(s) through the action of body fluids, enzymes, cells, and the like.

The term “function and/or characteristic of a cell” can refer to the modulation, growth, and/or proliferation of at least one cell, such as a progenitor cell and/or differentiated cell, the modulation of the state of differentiation of at least one cell, and/or the induction of a pathway in at least one cell, which directs the cell to grow, proliferate, and/or differentiate along a desired pathway, e.g., leading to a desired cell phenotype, cell migration, angiogenesis, apoptosis, etc.

The term “gel” includes gels and hydrogels.

The term “macromer” can refer to any natural or synthetic polymer or oligomer.

The term “polynucleotide” can refer to oligonucleotides, nucleotides, or to a fragment of any of these, to DNA or RNA (e.g., mRNA, rRNA, siRNA, tRNA) of genomic or synthetic origin which may be single-stranded or double-stranded and may represent a sense or antisense strand, to peptide nucleic acids, or to any DNA-like or RNA-like material, natural or synthetic in origin, including, e.g., iRNA, ribonucleoproteins (e.g., iRNPs). The term can also encompass nucleic acids (i.e., oligonucleotides) containing known analogues of natural nucleotides, as well as nucleic acid-like structures with synthetic backbones.

The term “polypeptide” can refer to an oligopeptide, peptide, polypeptide, or protein sequence, or to a fragment, portion, or subunit of any of these, and to naturally occurring or synthetic molecules. The term “polypeptide” can also include amino acids joined to each other by peptide bonds or modified peptide bonds, i.e., peptide isosteres, and may contain any type of modified amino acids. The term “polypeptide” can also include peptides and polypeptide fragments, motifs and the like, glycosylated polypeptides, and all “mimetic” and “peptidomimetic” polypeptide forms.

The term “cell” can refer to any progenitor cell, such as totipotent stem cells, pluripotent stem cells, and multipotent stem cells, as well as any of their lineage descendant cells, including more differentiated cells. The terms “stem cell” and “progenitor cell” are used interchangeably herein. The cells can derive from embryonic, fetal, or adult tissues. Examples of progenitor cells can include totipotent stem cells, multipotent stem cells, mesenchymal stem cells (MSCs), neuronal stem cells, hematopoietic stem cells, pancreatic stem cells, cardiac stem cells, embryonic stem cells, embryonic germ cells, neural crest stem cells, kidney stem cells, hepatic stem cells, lung stem cells, hemangioblast cells, and endothelial progenitor cells. Additional exemplary progenitor cells can include de-differentiated chondrogenic cells, chondrogenic cells, cord blood stem cells, multi-potent adult progenitor cells, myogenic cells, osteogenic cells, tendogenic cells, ligamentogenic cells, adipogenic cells, and dermatogenic cells.

The term “subject” can refer to any animal, including, but not limited to, humans and non-human animals (e.g., rodents, arthropods, insects, fish (e.g., zebrafish)), non-human primates, ovines, bovines, ruminants, lagomorphs, porcines, caprines, equines, canines, felines, ayes, etc.), which is to be the recipient of a particular treatment. Typically, the terms “patient” and “subject” are used interchangeably herein in reference to a human subject.

The term “tissue” can refer to an aggregate of cells having substantially the same function and/or form in a multicellular organism. “Tissue” is typically an aggregate of cells of the same origin, but may be an aggregate of cells of different origins. The cells can have the substantially same or substantially different function, and may be of the same or different type. “Tissue” can include, but is not limited to, an organ, a part of an organ, bone, cartilage, skin, neuron, axon, blood vessel, cornea, muscle, fascia, brain, prostate, breast, endometrium, lung, pancreas, small intestine, blood, liver, testes, ovaries, cervix, colon, stomach, esophagus, spleen, lymph node, bone marrow, kidney, peripheral blood, embryonic, or ascite tissue.

The terms “inhibit,” “silencing,” and “attenuating” can refer to a measurable reduction in expression of a target mRNA (or the corresponding polypeptide or protein) as compared with the expression of the target mRNA (or the corresponding polypeptide or protein) in the absence of an interfering RNA molecule of the present invention. The reduction in expression of the target mRNA (or the corresponding polypeptide or protein) is commonly referred to as “knock-down” and is reported relative to levels present following administration or expression of a non-targeting control RNA.

The term “population” can refer to a collection of cells, such as a collection of progenitor and/or differentiated cells.

The term “differentiated” as it relates to the cells can refer to cells that have developed to a point where they are programmed to develop into a specific type of cell and/or lineage of cells. Similarly, “non-differentiated” or “undifferentiated” as it relates to the cells can refer to progenitor cells, i.e., cells having the capacity to develop into various types of cells within a specified lineage.

Embodiments described herein relate to cytocompatible hydrogels, methods of forming the hydrogels, and to their use in regenerative medicine, cell-based technologies, drug delivery, and tissue engineering applications. The cytocompatible hydrogels include a plurality first polymer macromers crosslinked with a plurality of second polymer macromers different than the first polymer macromers by a plurality of β-hydroxythio-ether linkages. A plurality of cells and, optionally, bioactive agents can be encapsulated in the hydrogel.

The hydrogels described herein can be substantially cytocompatible (i.e., substantially non-cytotoxic) and include controllable physical properties, such as degradation rate, swelling behavior, and mechanical properties. Advantageously, encapsulated cells, such as hMSCs, can maintain their long-term high viability in the cytocompatible hydrogels. The hydrogel upon degradation, can produce substantially non-toxic products. Advantageously, the cytocompatible hydrogel can readily support three dimensional cell culture and maintain long-term high viability of the encapsulated cells, such as human mesenchymal stem cells (hMSCs), and provide a platform for delivery of bioactive agents to the encapsulated cells or to tissue to which the hydrogel is administered.

In some embodiments, the first polymer macromers are crosslinked with the second polymer macromers by reacting a plurality of epoxy polymer macromers with a plurality thiolated polymer macromers in a basic aqueous solution. By epoxy polymer macromers and thiolated polymer macromers, it is meant polymer macromers that include respectively epoxy or thio groups and that are capable of reacting in an aqueous solution to crosslink the epoxy polymer macromers with thiolated polymer macromers and form a hydrogel. The polymer macromers of the epoxy polymer macromers and the thiolated polymer macromers can include any polymer macromer used in the formation of a cytocompatible hydrogel. Such polymer macromers can include, for example, chitosan, poly(ethylene oxide), poly (lactic acid), poly(acrylic acid), poly(vinyl alcohol), poly(urethane), poly(N-isopropyl acrylamide), poly(vinyl pyrrolidone) (PVP), poly (methacrylic acid), poly(p-styrene carboxylic acid), poly(p-styrenesulfonic acid), poly(vinylsulfonicacid), poly(ethyleneimine), poly(vinylamine), poly(anhydride), poly(L-lysine), poly(L-glutamic acid), poly(gamma-glutamic acid), poly(carprolactone), polylactide, poly(ethylene), poly(ethylene glycol), poly(propylene), poly(glycolide), poly(lactide-co-glycolide), poly(amide), poly(hydroxylacid), poly(sulfone), poly(amine), poly(saccharide), poly(HEMA), poly(anhydride), collagen, gelatin, glycosaminoglycans (GAG), poly (hyaluronic acid), poly(sodium alginate), alginate, hyaluronan, agarose, polyhydroxybutyrate (PHB), and combinations thereof.

In other embodiments, the polymer macromers can include at least one of a straight chain or branched poly(ethylene glycol) (PEG) or an n-arm-poly(ethylene glycol), wherein n=3, 4, 5, 6, 7, 8, 10, or more, such as 3, 4, 6, 8, 10, or more. Epoxy or thiolated polymer macromers of the polymer macromers can include, for example, epoxy poly(ethylene glycol) macromers and thiolated poly(ethylene glycol) macromers.

In some embodiments, the thiolated poly(ethylene glycol) macromers can be n-arm-poly(ethylene glycol-thiol)macromers, wherein n is 3 or more, and the epoxy poly(ethylene glycol) macromers can be diepoxy poly(ethylene glycol) macromers. A cytocompatible hydrogel can be formed from the n-arm-poly(ethylene glycol-thiol)macromers and diepoxy poly(ethylene glycol) macromers by mixing solutions of n-arm-poly(ethylene glycol-thiol)macromers and diepoxy poly(ethylene glycol) in PBS (pH 7.4). The stoichiometric ratio of thiol to epoxy groups of the of n-arm-poly(ethylene glycol-thiol)macromers and diepoxy poly(ethylene glycol) can be about 1:1 to obtain a desired polymer concentration for reaction and gelation. A small amount of base (e.g., 0.2N NaOH (3 μL/50 μL gel)) can be added to the mixture to facilitate reaction of the of n-arm-poly(ethylene glycol-thiol)macromers and diepoxy poly(ethylene glycol). The mixture can be incubated at room temperature (RT) for a time (e.g., 30 min) to achieve gelation.

In some embodiment, he cells encapsulated in the hydrogel can be autologous, xenogeneic, allogeneic, and/or syngeneic. Where the cells are not autologous, it may be desirable to administer immunosuppressive agents in order to minimize immunorejection. The cells employed may be primary cells, expanded cells, or cell lines, and may be dividing or non-dividing cells. Cells may be expanded ex vivo prior to introduction into or onto the hydrogel. For example, autologous cells can be expanded in this manner if a sufficient number of viable cells cannot be harvested from the host subject. Alternatively or additionally, the cells may be pieces of tissue, including tissue that has some internal structure. The cells may be primary tissue explants and preparations thereof, cell lines (including transformed cells), or host cells.

Generally, cells can be introduced into the cytocompatible hydrogels in vitro, during gelation of the hydrogel. Cells may be mixed with the macromers used to form the hydrogel and cultured in an adequate growth (or storage) medium to ensure cell viability. If the cytocompatible hydrogel is to be implanted for use in vivo after in vitro seeding, for example, sufficient growth medium may be supplied to ensure cell viability during in vitro culture prior to in vivo application. Once the cytocompatible hydrogels have been implanted, the nutritional requirements of the cells can be met by the circulating fluids of the host subject.

Any available method may be employed to introduce the cells into the cytocompatible hydrogels. For example, cells may be injected into the cytocompatible hydrogels (e.g., in combination with growth medium) or may be introduced by other means, such as pressure, vacuum, osmosis, or manual mixing. Alternatively or additionally, cells may be layered on the cytocompatible hydrogels, or the cytocompatible hydrogels may be dipped into a cell suspension and allowed to remain there under conditions and for a time sufficient for the cells to incorporate within or attach to the cytocompatible hydrogel. Generally, it is desirable to avoid excessive manual manipulation of the cells in order to minimize cell death during the impregnation procedure. For example, in some situations it may not be desirable to manually mix or knead the cells with the cytocompatible hydrogels; however, such an approach may be useful in those cases in which a sufficient number of cells will survive the procedure. Cells can also be introduced into the cytocompatible hydrogels in vivo simply by placing the cytocompatible hydrogel in the subject adjacent a source of desired cells.

As those of ordinary skill in the art will appreciate, the number of cells to be introduced into the cytocompatible hydrogels will vary based on the intended application of the hydrogel and on the type of cell used. Where dividing autologous cells are being introduced by injection or mixing into the hydrogel, for example, a lower number of cells can be used. Alternatively, where non-dividing cells are being introduced by injection or mixing into the hydrogel, a larger number of cells may be required. It should also be appreciated that the cytocompatible hydrogel can be in either a hydrated or lyophilized state prior to the addition of cells. For example, the cytocompatible hydrogel can be in a lyophilized state before the addition of cells is done to re-hydrate and populate the hdyro with cells.

In other embodiments, the cytocompatible hydrogels can include at least one attachment molecule to facilitate attachment of at least one cell thereto. The attachment molecule can include a polypeptide or small molecule, for example, and may be chemically immobilized onto the cytocompatible hydrogel to facilitate cell attachment. Examples of attachment molecules can include fibronectin or a portion thereof, collagen or a portion thereof, polypeptides or proteins containing a peptide attachment sequence (e.g., arginine-glycine-aspartate sequence) (or other attachment sequence), enzymatically degradable peptide linkages, cell adhesion ligands, growth factors, degradable amino acid sequences, and/or protein-sequestering peptide sequences.

In other embodiments, the cytocompatible hydrogel can include at least one bioactive agent. The at least one bioactive agent can include any agent capable of modulating a function and/or characteristic of a cell that is dispersed on or within the cytocompatible hydrogel. Alternatively or additionally, the bioactive agent may be capable of modulating a function and/or characteristic of an endogenous cell surrounding the cytocompatible hydrogel implanted in a tissue defect, for example, and guide the cell into the defect.

Examples of bioactive agents include chemotactic agents, various proteins (e.g., short term peptides, bone morphogenic proteins, collagen, glycoproteins, and lipoprotein), cell attachment mediators, biologically active ligands, integrin binding sequence, various growth and/or differentiation agents and fragments thereof (e.g., EGF), HGF, VEGF, fibroblast growth factors (e.g., bFGF), PDGF, insulin-like growth factor (e.g., IGF-I, IGF-II) and transforming growth factors (e.g., TGF-β I-III), parathyroid hormone, parathyroid hormone related peptide, bone morphogenic proteins (e.g., BMP-2, BMP-4, BMP-6, BMP-7, BMP-12, BMP-13, BMP-14), sonic hedgehog, growth differentiation factors (e.g., GDF5, GDF6, GDF8), recombinant human growth factors (e.g., MP-52 and the MP-52 variant rhGDF-5), cartilage-derived morphogenic proteins (CDMP-1, CDMP-2, CDMP-3), small molecules that affect the upregulation of specific growth factors, polynucleotides, DNA fragments, DNA plasmids, MMPs, TIMPs, interfering RNA molecules, such as siRNAs, DNA encoding for an shRNA of interest, oligonucleotides, proteoglycans, glycoproteins, and glycosaminoglycans.

In other embodiments, the bioactive can include, for example, at least one of a peptide, protein, polynucleotide, or small molecule that modulates a function and/or characteristic of at least one of the cells. The polynucleotide can include DNA fragments, DNA plasmids, and/or interfering RNA molecules that modulates a function and/or characteristic of at least one of the cells.

In some embodiments, the interfering RNA molecule can include any RNA molecule that is capable of silencing a target mRNA and thereby reducing or inhibiting expression of a polypeptide encoded by the target mRNA. Alternatively, the interfering RNA molecule can include a DNA molecule encoding for a shRNA of interest. For example, the interfering RNA molecule can comprise a short interfering RNA (siRNA) or microRNA molecule capable of silencing a target mRNA that encodes any one or combination of the polypeptides or proteins described above. In one example, the interfering RNA molecules can include pro-osteogenic siNoggin. Pro-osteogenic siNoggin was found to substantially enhance the osteogenesis of hMSCs encapsulated in the hydrogel.

The bioactive agent, including interfering RNA, such as siNoggin, can be provided in at least one carrier material capable of carrying and differentially and/or controllably releasing at least one bioactive agent to the cell or within the cells. Carrier materials can be directly linked to the bioactive agent and/or physically associated with the bioactive agent. Carrier materials can include a variety of known microparticles or nanoparticles including, for example, polymer-based and calcium phosphate-based microparticles and nanoparticles. It will be appreciated that a carrier molecule, such as a positively-charged polymer (e.g., PEI) can be included along with a desired bioactive agent (e.g., a DNA plasmid encoding an siRNA or siRNA molecule).

Polymer-based carrier materials can include a biodegradable polymer capable of controllably and/or differentially releasing at least one bioactive agent. For example, a polymer-based carrier material can be a biodegradable polymer in microparticle form. Microparticles can have a diameter less than 1 mm and typically between 1 and 200 microns. Microparticles can include both microspheres and microcapsules, and may have an approximately spherical geometry and be of fairly uniform size. Microspheres can have a homogeneous composition, and microcapsules can include a core composition (e.g., a bioactive agent) distinct from a surrounding shell. For the purposes of the present invention, the terms “microsphere,” “microparticle,” and “microcapsule” may be used interchangeably.

In some embodiment, at least one carrier material can differentially or controllably release the at least one interfering RNA molecule or be taken up (e.g., via endocytosis) by at least one cell to modulate a function and/or characteristic of the at least one cell. The at least one interfering RNA molecule may be at least partially coated on the surface of the at least one carrier material or, alternatively, dispersed, incorporated, and/or impregnated within the at least one carrier material. For example, a carrier material comprising a PLGA microparticle can be impregnated with an siRNA molecule capable of targeting an mRNA corresponding to at least a portion of the noggin gene. Alternatively, a carrier material comprising a PEI microparticle or nanoparticle can be impregnated with an siRNA molecule capable of targeting an mRNA corresponding to noggin. It will be appreciated that the carrier material can be coated with a polynucleotide and/or polypeptide to prevent or reduce aggregation and/or promote cellular uptake of the carrier material. It will also be appreciated that the carrier material can include the same or different interfering RNA molecules, and that two, three, or even more carrier materials can be included with the same or different interfering RNA molecules in the bioresorbable implant composition.

In other embodiments, the at least one bioactive agent incorporated on or within the at least one carrier material can comprise first and second bioactive agents respectively incorporated on or within first and second carrier materials. The first and second carrier materials may comprise the same or different materials. Additionally, the first and second bioactive agents may comprise the same or different agents. The first and second carrier materials can differentially, sequentially, and/or controllably release the first and second bioactive agents to modulate a different function and/or characteristic of at least one cell. It will be appreciated that the first carrier material can release the first bioactive agent with a different release profile than the release profile of the second bioactive agent from the second carrier material. Additionally, it will be appreciated that the first carrier material can degrade or diffuse before the degradation or diffusion of the second carrier material or allow for an increased rate of release or diffusion of the first bioactive agent compared to the release of the second bioactive agent. The first and second carrier materials may be dispersed uniformly within the hydrogel or, alternatively, dispersed such that different densities of carrier materials are dispersed within different portions of the hydrogel.

The hydrogels described herein can be used in a variety of biomedical applications, including tissue engineering, drug delivery applications, and regenerative medicine. In one example, the hydrogel can be used to promote tissue growth in a subject. One step of the method can include identifying a target site. The target site can comprise a tissue defect (e.g., cartilage and/or bone defect) in which promotion of new tissue (e.g., cartilage and/or bone) is desired. The target site can also comprise a diseased location (e.g., tumor). Methods for identifying tissue defects and disease locations are known in the art and can include, for example, various imaging modalities, such as CT, MRI, and X-ray.

The tissue defect can include a defect caused by the destruction of bone or cartilage. For example, one type of cartilage defect can include a joint surface defect. Joint surface defects can be the result of a physical injury to one or more joints or, alternatively, a result of genetic or environmental factors. Most frequently, but not exclusively, such a defect will occur in the knee and will be caused by trauma, ligamentous instability, malalignment of the extremity, meniscectomy, failed aci or mosaicplasty procedures, primary osteochondritis dessecans, osteoarthritis (early osteoarthritis or unicompartimental osteochondral defects), or tissue removal (e.g., due to cancer). Examples of bone defects can include any structural and/or functional skeletal abnormalities. Non-limiting examples of bone defects can include those associated with vertebral body or disc injury/destruction, spinal fusion, injured meniscus, avascular necrosis, cranio-facial repair/reconstruction (including dental repair/reconstruction), osteoarthritis, osteosclerosis, osteoporosis, implant fixation, trauma, and other inheritable or acquired bone disorders and diseases.

Tissue defects can also include cartilage defects. Where a tissue defect comprises a cartilage defect, the cartilage defect may also be referred to as an osteochondral defect when there is damage to articular cartilage and underlying (subchondral) bone. Usually, osteochondral defects appear on specific weight-bearing spots at the ends of the thighbone, shinbone, and the back of the kneecap. Cartilage defects in the context of the present invention should also be understood to comprise those conditions where surgical repair of cartilage is required, such as cosmetic surgery (e.g., nose, ear). Thus, cartilage defects can occur anywhere in the body where cartilage formation is disrupted, where cartilage is damaged or non-existent due to a genetic defect, where cartilage is important for the structure or functioning of an organ (e.g., structures such as menisci, the ear, the nose, the larynx, the trachea, the bronchi, structures of the heart valves, part of the costae, synchondroses, enthuses, etc.), and/or where cartilage is removed due to cancer, for example.

After identifying a target site, such as a cranio-facial cartilage defect of the nose, the hydrogel can be administered to the target site. The hydrogel can be prepared by mixing plurality of epoxy polymer macromers and a plurality thiolated polymer macromers in a basic aqueous solution. A plurality of cells, such as chondrocytes or hMSCs, can be loaded in the hydrogel during gelation. Chondrocytes and/or hMSCs may be obtained from a host subject and then expanded to a desired density ex vivo.

Next, the hydrogel may be loaded into a syringe or other similar device and injected or implanted into the tissue defect. Upon injection or implantation into the tissue defect, the hydrogel be formed into the shape of the tissue defect using tactile means.

After implanting the hydrogel into the subject, the chondrocytes or hMSCs can begin to migrate from the hydrogel into the tissue defect, express growth and/or differentiation factors, and/or promote chondroprogenitor cell expansion and differentiation.

The following example is for the purpose of illustration only and are not intended to limit the scope of the claims, which are appended hereto.

### Example

This Example describes a cytocompatible PEG-based hydrogel system formed via thiol-epoxy “click” chemistry in aqueous media that was prepared under mild conditions directly from commercially available macromers and possessed the capability of encapsulating hMSCs, maintaining high cell viability, and supporting cell differentiation. In addition, a pro-osteogenic siNog (sequence 5′-AAC ACU UAC ACU CGG AAA UGA UGG G-3′) (SEQ ID NO: 1) was loaded into the hydrogels to demonstrate the potential to guide the differentiation of hMSCs encapsulated in this hydrogel system.

### Materials and Methods

Poly(ethylene glycol)-di(glycidyl ether) (PEG-DE, MW 500 Da) and Alizarin Red S (ARS) were purchased from Sigma Aldrich (St Louis, Mo.). 8-arm PEG-thiol (PEG(-SH)₈, MW 10 kDa) and 4-arm PEG-thiol (PEG(-SH)₄, MW 5 kDa) were purchased from Jenkem Technology USA (Allen, Tex.).

siRNA against Noggin (siNog, sequence 5′-AAC ACU UAC ACU CGG AAA UGA UGG G-3′) (SEQ ID NO: 2) and non-targeting negative control siRNA (siCT, sequence 5′-UUC UCC GAA CGU GUC ACG UTT-3′) (SEQ ID NO: 3) were purchased from Insight Genomics (Falls Church, Va.).

Low-glucose DMEM (DMEM-LG) was purchased from Sigma. Phosphate-buffered saline (PBS), prescreened fetal bovine serum (FBS, Gibco), and penicillin/streptomycin (P/S) were obtained from Fisher Scientific. hMSCs were isolated, stored under liquid nitrogen, plated at 5,000 cells/cm² and cultured for 10 to 14 days at 37° C. and 5% CO₂ in a humidified incubator before harvesting for the experiments.

The data is presented as mean±standard deviation. Statistical comparisons were performed with Tukey-Kramer Multiple Comparisons Test with one-way analysis of variance (ANOVA) using InStat software (GraphPad Software, La Jolla, Calif.). p<0.05 was considered statistically significant.

### Hydrogel Preparation

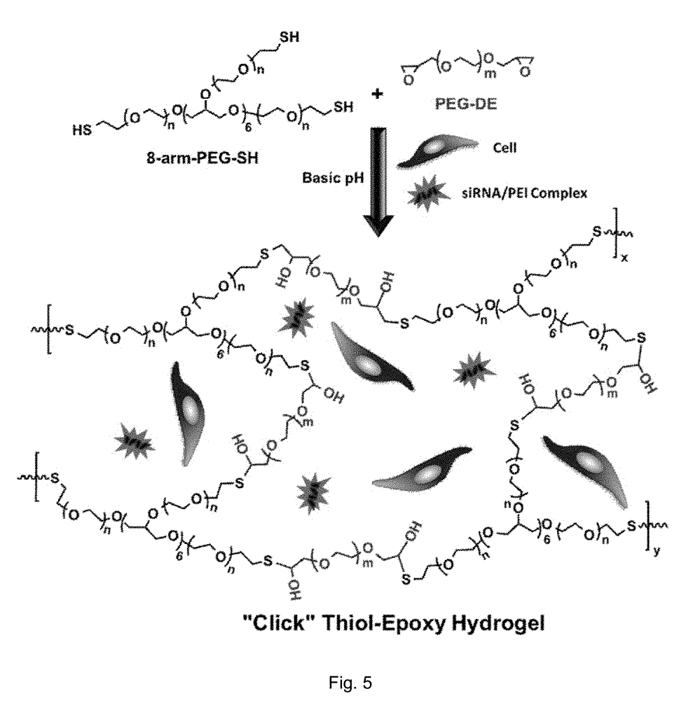

The hydrogels were fabricated using thiol-epoxy click chemistry. Solutions of PEG-epoxy and PEG-thiol in PBS (pH 7.4) were mixed at a 1:1 stoichiometric ratio of thiol to epoxy groups to obtain the desired polymer concentration. A small amount of 0.2N NaOH (3 μL/50 μL gel) was added to the mixture of the polymer solution. The mixture was vortexed for 10 s, then placed between two glass-plates with spacers and incubated at room temperature (RT) for 30 min to achieve gelation. FIG. 5 shows a schematic of thiol-epoxy “click” hydrogel formation with encapsulation of hMSCs and siRNA/PEI complexes for tissue regeneration applications.

### Reaction Monitoring Via NMR Measurement

The reaction between the thiol and epoxy groups of the macromers in aqueous media was monitored using a 500 MHz NMR instrument (Bruker Ascend III, Bruker BioSpin Corp., Billerica, Mass.) equipped with a prodigy probe. Proton and carbon NMR were recorded using 1 and 3% polymer solutions in D₂O, respectively.

Proton NMR confirmed that the reaction between thiol and epoxy groups did not occur in aqueous media at neutral pH (Figure S1A). However, in the presence of a small amount of NaOH, the reaction took place and new peaks representing the protons of formed β-hydroxyl thioether groups were recorded after 2 min of mixing. The proton and carbon NMR spectrum also confirmed that the individual macromers in solution did not self-react at either neutral or basic pH.

### Gelation Time Via Tube Inverting Method

To determine the gelation time of the hydrogels, the tube inverting method was carried out at room temperature. Briefly, the solution of polymers and NaOH at desired polymer concentrations (total 100 μL) was mixed in a 1.7 mL microcentrifuge tube and vortexed for 10 s; then, the gelation time was measured (N=3) at room temperature (RT). The gelation time is the period from when all of the materials are mixed until the point when the mixture solution stops flowing when the tubes are inverted by 180°.

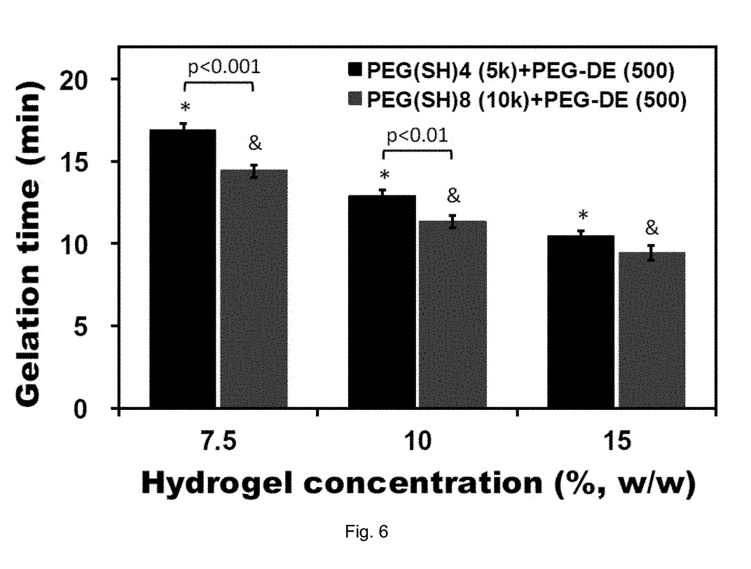

The gelation time of the hydrogels ranged from 9.4 to 16.9 min depending on the macromer structures and hydrogel concentrations (FIG. 6). Gelation time increased with decreasing polymer concentration or average functional groups per macromer molecule.

### Rheological Measurement to Examine Gelation Time

The storage (G′) and loss (G″) moduli of the mixed macromer solutions used to form hydrogels were measured using a dynamic Haake Mars III Rotational Rheometer (Thermo Fisher Scientific Inc., Waltham, Mass.). The macromer mixtures were prepared and then placed between two stainless steel parallel plates (8.0 mm diameter upper plate; 50 mm diameter lower plate) with a gap of 1.0 mm. The measurement was performed at 25° C. using oscillation time sweep mode with a controlled stress of 0.4 Pa and a frequency of 1.0 rad/s.

### Swelling of Hydrogel

To determine the swelling ratio of prepared hydrogels, the hydrogels were prepared as described previously, rinsed 3 times in an excessive amount of PBS (pH 7.4) for 10 min, rinsed in deionized water (diH₂O) overnight at 4° C., frozen at −80° C. for at least 4 h, and lyophilized. Then, the initial dried weights (W_{do}) were measured. The dried hydrogels were rehydrated by immersion into PBS (pH 7.4) or DMEM-HG (10 mL) and then incubated at 37° C. The PBS and media were changed every three days. At designated time points, the hydrogels were collected, the wet weights (W_{wt}) were measured and the swelling ratios were calculated with the following equation: (W_{wt}−W_{do})/W_{do}*100.

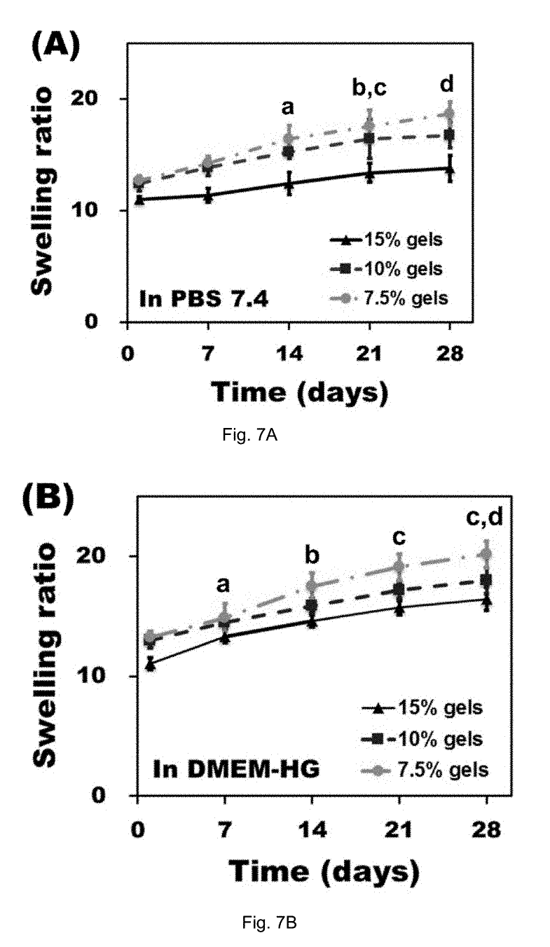

The swelling ratio of hydrogels in DMEM-HG is slightly higher than that in PBS (FIG. 7), which may result the differences in composition between the two solutions, such as glucose content. Hydrogel swelling ratio slightly increased over time and increased with decreasing hydrogel concentration.

siRNA/PEI Nanocomplexes Preparation

siRNA was diluted in RNase-free PBS (pH 7.4) to prepare a 100 μM stock solution. PEI was dissolved in RNase-free water at a stock concentration of 1 mg/mL (40 μM). siRNA/PEI complexes were prepared at an N/P ratio of 11.5, where N and P refer to the number of amine and phosphate groups in PEI and siRNA, respectively. To prepare the siRNA/PEI complexes, siRNA (6.4 μM) and PEI (4.8 μM) solutions were prepared separately in PBS. The siRNA solution was then added to the PEI solution (1/1, v/v) and the mixture was vortexed for 30 s. Then, the mixture was incubated at room temperature for 30 min to form the complexes before loading into the hydrogel.

### Cell Encapsulation, Viability, and Differentiation

To encapsulate hMSCs in the hydrogels, the mixture of PEG-DE (MW 500 Da) and PEG(-SH)₈ (MW 10 kDa) (Epoxy/SH=1/1, mol/mol) at basic pH was prepared and incubated at RT for 7 min and then the hMSCs (5 E6 cells/mL) and siRNA/PEI complexes, if needed, were loaded. Non-targeting siRNA (siCT) and siNog were used as control and pro-osteogenic RNA groups, respectively. The mixture was then placed between 2 glass plates with 600 μm spacers and gelation occurred for an additional 10 min. The formed hydrogel sheets were rinsed 3 times with excessive PBS pH 7.4 (10 mL) for 10 min and punched into 3.5 or 5.0 mm gel disks. The gel disks were then transferred to wells of 24-well plates for further experiments.

To examine cell viability, the gel disks were transferred into wells of 24-well plates containing 0.5 mL normal growth media (DMEM-LG supplemented with 10% FBS and 1% P/S), which was replaced twice per week. At designated time points, live-dead staining was performed to determine the viability of hMSCs encapsulated within the hydrogels using fluorescein diacetate (FDA, Sigma), which stains the cytoplasm of viable cells green, and ethidium bromide (EB, Sigma), which stains the nuclei of nonviable cells orange-red. 20 μL of live/dead staining solution, which was freshly prepared by mixing 1 mL FDA solution (1.5 mg/mL in dimethyl sulfoxide (Sigma)) and 0.5 mL EB solution (1 mg/mL in PBS) with 0.3 mL PBS (pH 8.0), was added to each well containing the hMSC-hydrogel constructs. After 5 min incubation at room temperature, photomicrographs of the samples were obtained using an ECLIPSE TE 300 (Nikon, Tokyo, Japan) fluorescence microscope equipped with a Retiga-SRV digital camera (Qimaging, Burnary, BC, Canada). Cell viability was then quantified using Fiji software (ref. S8) and photomicrographs of live/dead stained samples (N>=3 images/group).

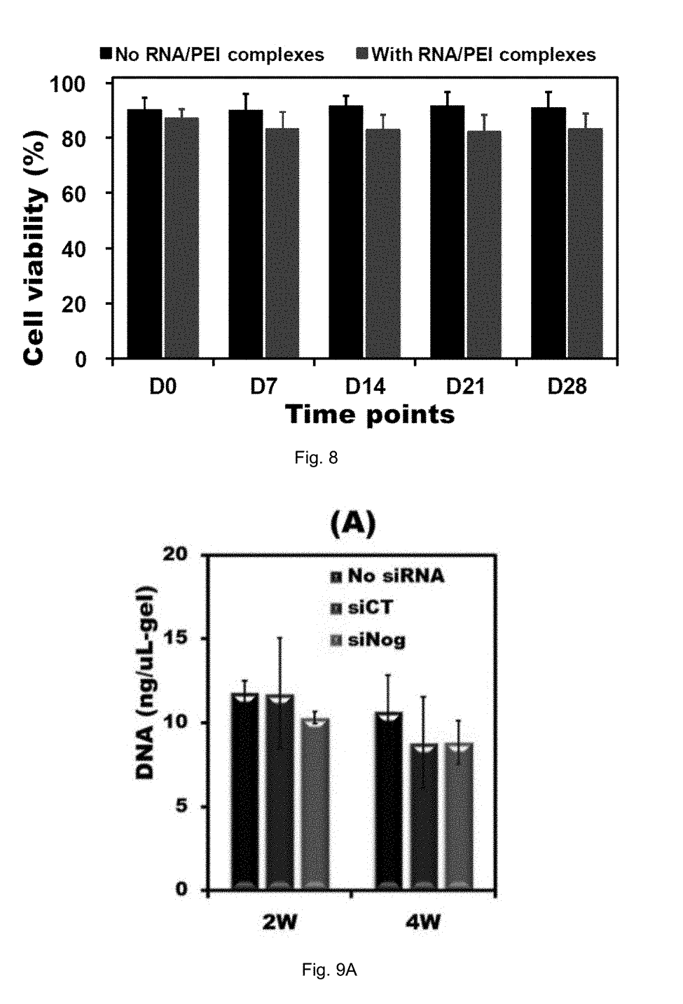

Although there may be limitations in the quantification accuracy of the live/dead images due to some cell clusters observed in the gels, there were no significant differences in cell viability in between experimental time points or between groups with and without loaded RNA/PEI complexes in the hydrogels (FIG. 8).

To examine the osteogenic differentiation of encapsulated hMSCs, the gels disks were transferred into wells of 24-well plates containing 0.5 mL growth or osteogenic media (DMEM-LG supplemented with 10% FBS, 1% P/S, 100 nM dexamethasone (MP Biomedicals, Solon, Ohio), 10 mM β-glycerophosphate (CalBiochem, Billerica, Mass.) and 100 μM ascorbic acid (Wako USA, Richmond, Va.)), which was replaced twice a week. At specific time points, the hydrogels were harvested for biochemical assays or for staining of calcium (Ca) deposition in the hydrogels. To perform the biochemical assays, the gels were placed into 1 mL of CelLytic MT buffer (Sigma) and homogenized at 35,000 rpm for 60 s using a TH homogenizer (Omni International, Marietta, Ga.). Supernatants (400 μL) of the homogenized solutions were collected post-centrifugation at 500 g with a Sorval Legend RT plus centrifuge (Thermo Fisher Scientific Inc., Waltham, Mass.) for DNA quantification (N=3, each sample was assayed in duplicate). The Picogreen assay kit (Life Technologies) was used to quantify DNA from the supernatant on a plate reader (fmax, Molecular Devices) at excitation/emission of 485 nm/538 nm. A series of known DNA concentrations in CelLytic MT buffer was prepared from the Picogreen assay kit stock DNA solution and used to establish a standard curve to calculate sample DNA amount. To quantify the amount of deposited Ca from differentiated hMSCs, HCl 1.2N (equal volume with the remaining supernatant) was added to the remaining sample tubes (600 μL supernatant and pellet) to dissolve any undissolved mineralized Ca particles. This may slightly over-calculate the true calcium content. The sample tubes were then incubated overnight at 4° C. before the Ca content was measured using a Ca assay kit (Pointe Scientific, Canton, Mich.). Briefly, 4 μL of the samples or standard Ca solutions (concentration ranges from 0 to 250 ng/mL diluted in PBS from stock 1.0 g/L Ca solution (Sigma)) was added to the color and buffer reagent (200 μL) (Fisher Scientific) and the absorbance was measured at 570 nm with a plate reader (VersaMax, Molecular Devices, Sunnyvale, Calif.). To visualize the deposited Ca from differentiated hMSCs, the hydrogels were fixed with 4% neutral buffered formalin for 24 h and rinsed twice with PBS before embedding in optimal cutting temperature (OCT; Fisher Scientific) compound and flash-freezing in liquid nitrogen. The samples were stored at −20° C. prior to cryosectioning 20 μm thick sections. The hydrogel layers were transferred to glass sides, stained with ARS, and then mounted using coverslips with fluoromount (Sigma-Aldrich). The stained samples were imaged using an Olympus BX61VS microscope (Olympus, Center Valley, Pa.) with a Pike F-505 camera (Allied Vision Technologies, Stadtroda, Germany).

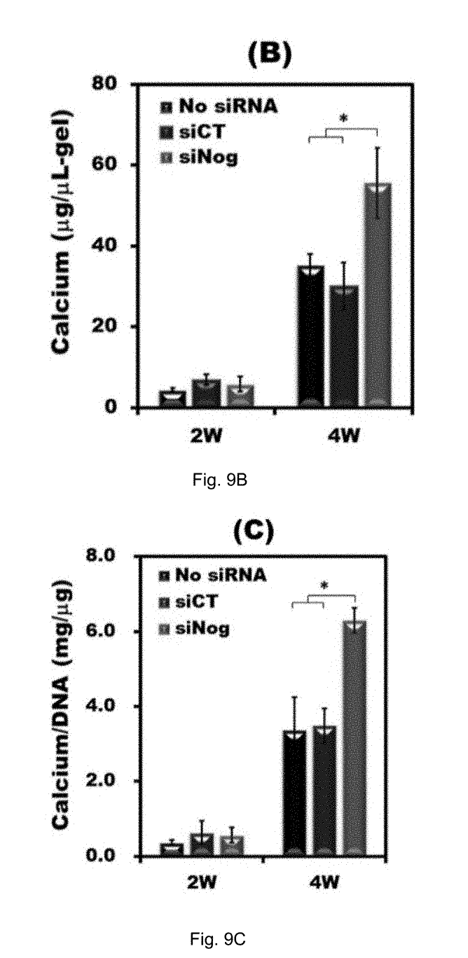

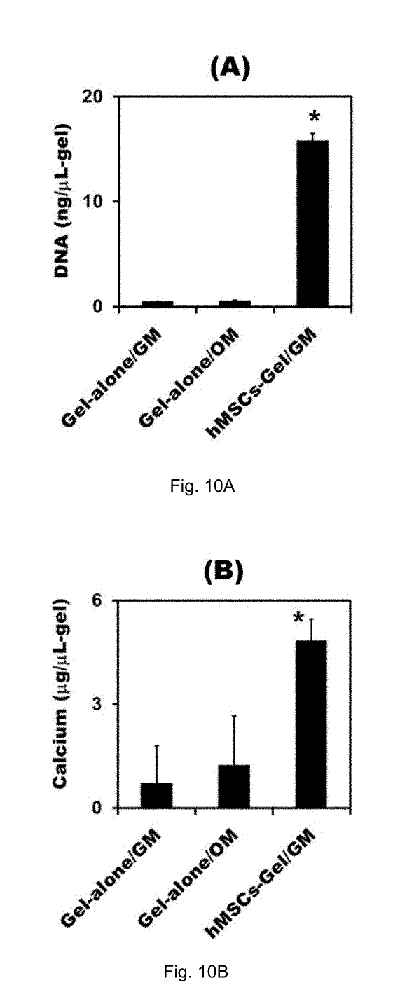

There was no difference in DNA content between groups did not contain RNA and groups encapsulated with either siCT or siNog (FIG. 9A), indicating low cytotoxicity with siRNA treatment. siCT did not impact Ca deposition at 4 weeks compared to the no siRNA group, while a significant increase in Ca deposition was found in the siNog loaded group compared to the controls at this time point (FIG. 9B).

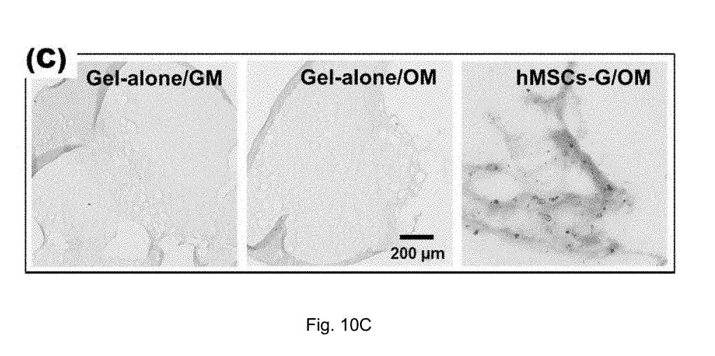

To confirm the measured Ca was produced by differentiated hMSCs and not simply ectopic calcification, empty gels (Gel-alone) cultured in both GM and OM for 4 weeks were biochemically and histologically evaluated (FIG. 10). Negligible background “DNA” was measured in Gel-alone groups (FIG. 10A). Less than 1.23 ng Ca/μL-gel was measured in Gel-alone groups compared to approximately 5 ng Ca/μL-gel in hMSCs-encapsulated hydrogels cultured in GM (hMSCs-Gel/GM) (FIG. 10B). No deposited Ca was found in the histological images of sectioned Gel-alone groups cultured in GM or OM for 4 weeks (FIG. 10C).

### Results

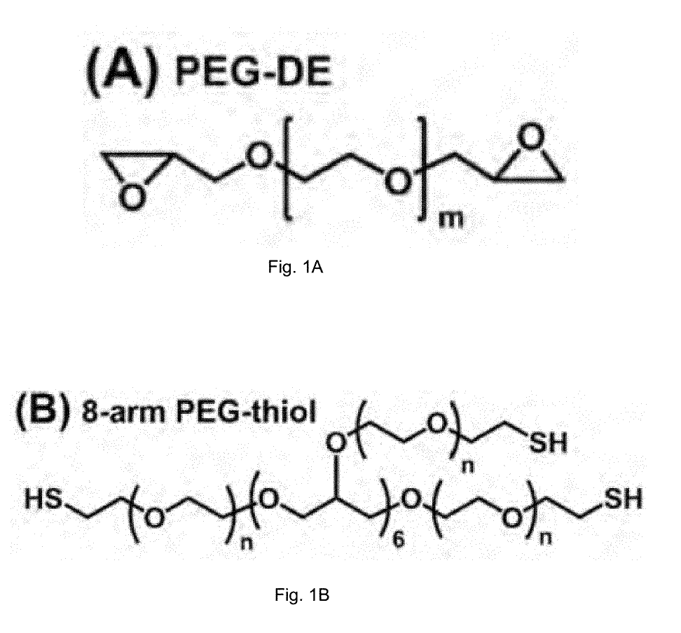

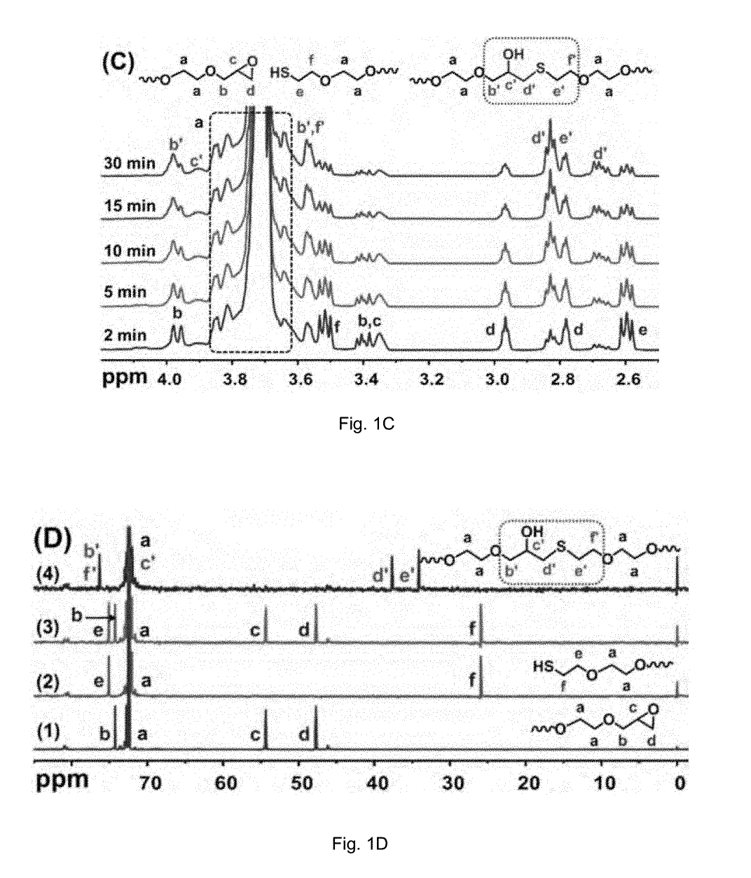

The hydrogels were fabricated by mixing solutions of 8-arm thiolated PEG (PEG(-SH)₈) and PEG-diepoxy (PEG-DE) (FIGS. 1 A and B, respectively) at basic pH. A detailed schematic of hydrogel preparation is shown in FIG. 5. In aqueous media at basic pH, the reaction between epoxy and thiol groups occurred rapidly, as demonstrated by the appearance and increase in intensity over time of methylene protons of the formed β-hydroxyl thioether groups in NMR spectra (FIG. 1C). Consistent data was also recorded via carbon NMR (FIG. 1D). However, the reaction did not occur at neutral pH, as shown via monitoring the change of chemical groups in the macromer mixture solution. In addition, the NMR confirmed that self-reaction of thiol or epoxy groups in the individual macromer solutions did not occur. These NMR results confirm that the reaction of thiol and epoxy groups in aqueous media at basic pH can be used to prepare a hydrogel network.

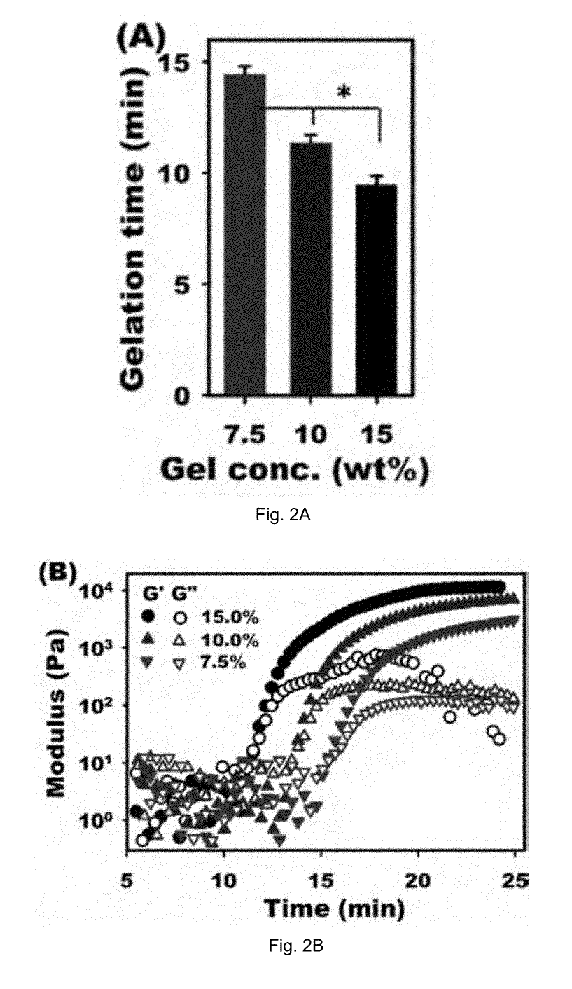

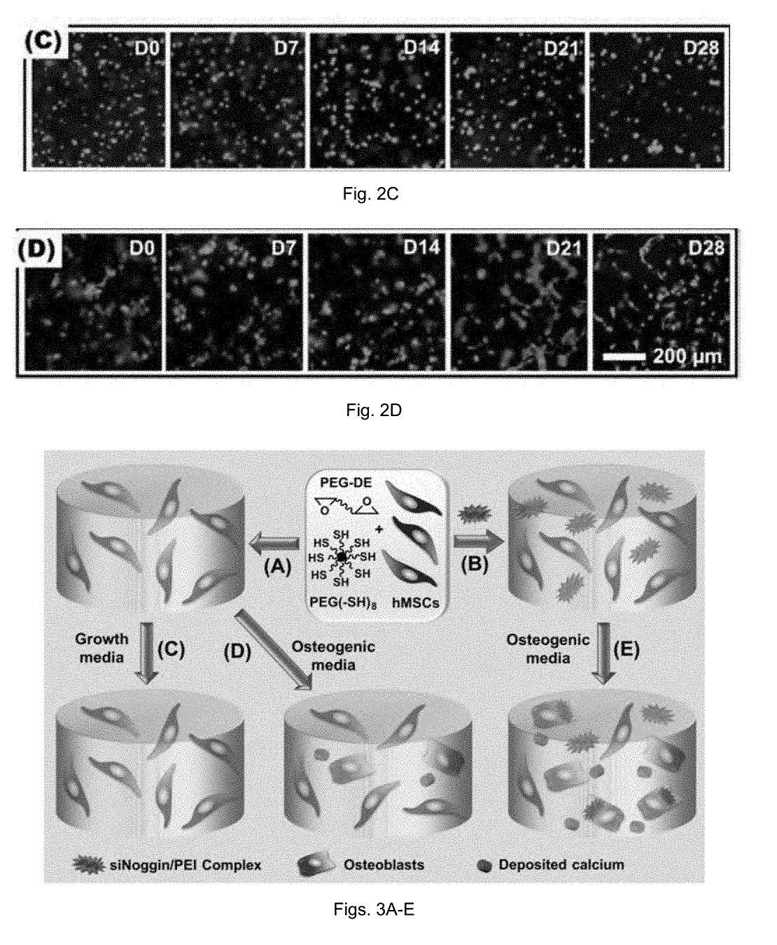

After confirming the reactivity between epoxy and thiol groups, the hydrogels were fabricated and the gelation times were measured using the tube inversion method and rheological measurements. It was observed in both methods that the gelation time increased with decreasing macromer concentration. The hydrogels formed between 9.4 to 16.9 min after formulation, depending on the final macromer concentration (FIG. 2A) and the number of arms present in the PEG-thiol (FIG. 6). Similar results were observed in rheological measurements, in which the gelation point was determined as the time at which G′ and G″ crossed each other (FIG. 2B). This gelation timeframe offers a suitable window, which ranges from minutes to an hour, for cell encapsulation within the hydrogel and subsequent injection into the body, which will be valuable for tissue engineering applications. Increasing the hydrogel concentration also led to increased hydrogel stiffness, as illustrated by increasing the G′. In addition, the swelling ratio of the hydrogels increased slightly over the course of 4 weeks in both PBS and DMEM-HG media (FIG. 7). A slightly higher swelling ratio in DMEM-HG group compared to those in PBS was observed which may potentially result from the differences in composition between the two media, such as glucose content. Decreasing crosslinking density via lowering hydrogel concentration led to the increasing in swelling ratio (FIG. 7).

Biocompatibility is a very important factor to consider in the engineering of biomaterial systems for cell encapsulation-based tissue regeneration. To determine the potential cytotoxicity of this hydrogel system, hMSCs were encapsulated in the hydrogels with or without loading of siRNA against luciferase (siLuc) complexed with polyethyleneimine (PEI), in the form of siRNA/PEI nanocomplexes. The cell viability over time was examined using live/dead staining. The encapsulated hMSCs remained highly viable over 4 weeks of culture, regardless of the absence or presence of siRNA/PEI nanocomplexes (FIG. 2C, D), indicating the cytocompatibility of the fabricated hydrogel system. In addition, using Fiji software to quantify the cell viability in live/dead stained images, no significant differences in were observed between experimental time points or between groups with and without loaded RNA/PEI complexes in the hydrogels (FIG. 8). Although a hydrogel fabricated via thiol-epoxy chemistry in aqueous media was recently confirmed to be biocompatible in the presence of monolayer cultured cells for up to 24 h, longer culture times and, importantly, cell encapsulation were not examined. The developed hydrogel system in this study offers the capacity to encapsulate cells with long-term viability, demonstrating its potential for 3D cell culture.

After confirming cytocompatibility of the hydrogel system, hMSCs were then encapsulated within hydrogels loaded with and without siNog, a pro-osteogenic siRNA, complexed with PEI to examine the ability of the developed hydrogel and/or siNog to support hMSC osteogenesis (FIG. 3). Calcium (Ca) is a major component of hydroxyapatite, the inorganic portion of bone extracellular matrix, and thus, was used to evaluate the degree of encapsulated hMSC osteogenic differentiation. It was hypothesized that hMSCs in the constructs cultured in growth media (GM) would survive but not differentiate (FIG. 3C), while encapsulated hMSCs cultured in osteogenic media (OM) would differentiate and deposit Ca (FIG. 3D). Additionally, it was anticipated that encapsulated siNog in the hydrogels would enhance hMSCs differentiation rate and extent, and, therefore, increase the amount of deposited Ca (FIG. 3E).

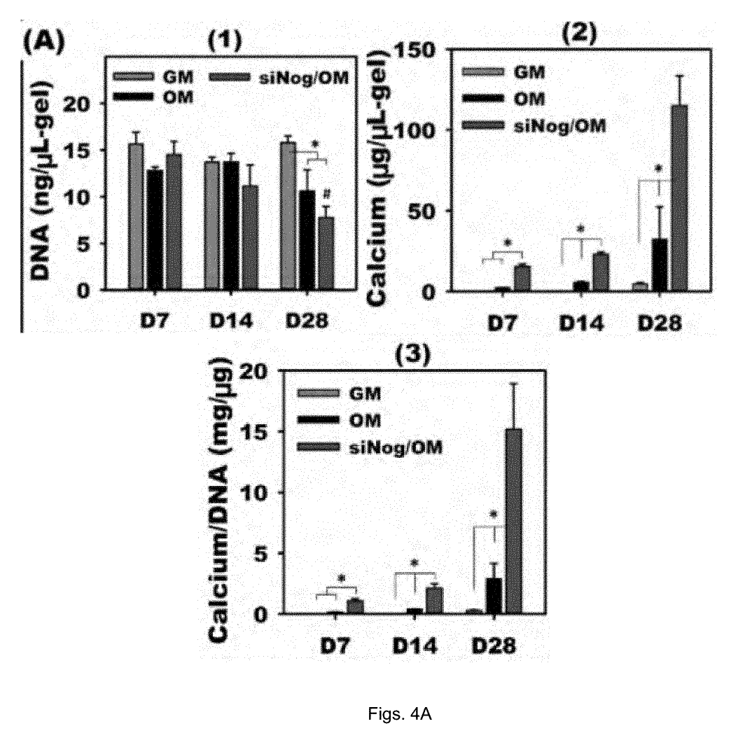

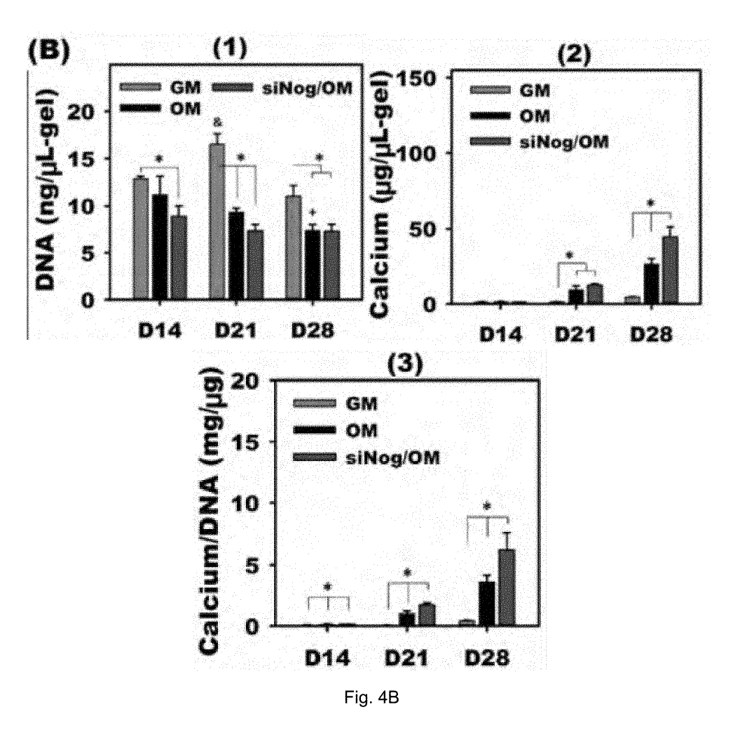

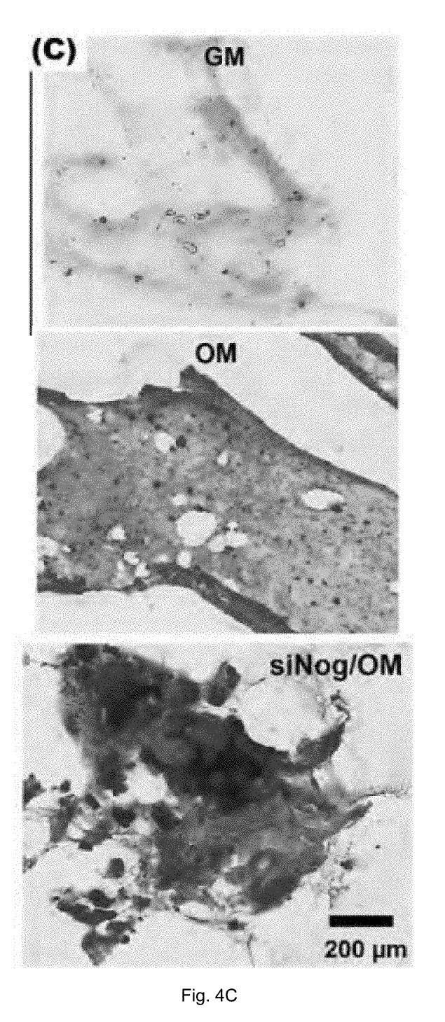

To validate the hypothesis, hMSCs from two different donors were separately encapsulated in the hydrogel constructs, which were then cultured in GM or OM, and harvested at pre-determined time points to assay DNA and Ca content (FIG. 4). DNA content was quantified as an indirect measure of construct cellularity, and Ca content served as a late differentiation marker for osteogenesis, respectively. In GM, no decrease in DNA content in RNA-free constructs was observed for either donor after 4 weeks of culture (FIG. 4A1, 4B1), indicating the cytocompatibility of the developed hydrogel system. When the RNA-free constructs were cultured in OM, DNA significantly decreased from 2 to 4 weeks in donor 2 constructs (FIG. 4B1), while a significant decrease in DNA was observed in both donors at 3 and/or 4 weeks compared to culturing in GM (FIG. 4A1, 4B1). There are reports in the literature that osteoblasts have a limited life span and tend to become lining cells or osteocytes or undergo apoptosis. Therefore, the decrease in DNA content observed may be attributed to a degree of apoptosis that occurred during the osteogenic differentiation of the encapsulated hMSCs. hMSCs encapsulated in hydrogels cultured in OM showed significantly more Ca deposition compared to those cultured in GM, both in the absolute amount and after normalizing to DNA content (FIG. 4A2,3, 4B2,3), demonstrating that this hydrogel system supports the osteogenesis of encapsulated hMSCs.

When cultured in OM, siRNA loading within hydrogels did not affect the DNA content of constructs from either donor (FIG. 4A1, B1 and FIG. 9), indicating low cytotoxicity with siRNA treatment. Importantly, loaded siNog significantly enhanced the osteogenesis of encapsulated hMSCs, as demonstrated by a significant increase in Ca deposition compared to hydrogels cultured in OM without incorporated genetic material (FIG. 4A2,3, 4B2,3) or loaded with non-targeting control siRNA (siCT; FIG. 9B, C in SI). siNog was previously reported to enhance the osteogenesis of hMSCs with approximately 3.5-fold and 1.5-to-2.2-fold increases in Ca deposition when cultured in monolayer or encapsulated in 3D hydrogel networks, respectively. Construct sections stained for calcium with Alizarin Red S (ARS) also exhibited more intense staining in siNog/OM constructs compared to the other groups (FIG. 4C), which is consistent with biochemical results. No Ca was found biochemically or histologically in hydrogels without cells and siRNA cultured in either GM or OM for 4 weeks (FIG. 10), indicating that the detected Ca in the previously described experiments was produced by differentiated hMSCs. These results confirm that the developed hydrogel system is a promising biomaterial candidate for 3D cell culture and bone tissue regeneration.

In summary, a novel PEG-based hydrogel formed via thiol-epoxy “click” reaction has been engineered. The hydrogel supports 3D cell culture and the osteogenesis of encapsulated hMSCs. Specifically, loading the developed hydrogels with siNog, a pro-osteogenic siRNA, significantly enhanced the osteogenic differentiation of hMSCs. This crosslinking strategy have great utility in forming hydrogels for cell encapsulation, tissue engineering, bioactive factor delivery and other biomedical applications.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention, we claim:

1. A composition comprising:
a cytocompatible hydrogel that includes a plurality first polymer macromers crosslinked with a plurality of second polymer macromers different than the first polymer macromers by a plurality of β-hydroxythio-ether linkages, and
a plurality of cells encapsulated in the hydrogel, the hydrogel upon degradation, produce substantially non-toxic products.

2. The composition of claim 1, wherein the first polymer macromers are crosslinked with the second polymer macromers by reacting a plurality of epoxy polymer macromers with a plurality thiolated polymer macromers in a basic aqueous solution.

3. The composition of claim 2, wherein the epoxy polymer macromers are epoxy poly(ethylene glycol) macromers and the thiolated polymer macromers are thiolated poly(ethylene glycol) macromers.

4. The composition of claim 3, wherein the thiolated poly(ethylene glycol) macromers are n-arm-poly(ethylene glycol-thiol)macromers, wherein n is 3 or more.

5. The composition of claim 3, wherein the epoxy poly(ethylene glycol) macromers are diepoxy poly(ethylene glycol) macromers.

6. The composition of claim 1, further comprising at least one bioactive agent.

7. The composition of claim 6, further comprising at least one carrier material dispersed on or within the hydrogel, the carrier material including the bioactive agent and releasing the bioactive agent to modulate a function and/or characteristic of at least one of the cells.

8. The composition of claim 6, wherein the bioactive agent comprising at least one of DNA fragments, DNA plasmids, and/or interfering RNA molecules that modulates a function and/or characteristic of at least one of the cells.

9. The composition of claim 8, the interfering RNA molecules comprising siRNA or miRNA.

10. The composition of claim 1, wherein the cells comprise progenitor cells, undifferentiated cells and/or differentiated cells.

11. The composition of claim 7, wherein the cells include mesenchymal stem cells.

12. A composition comprising:
a cytocompatible hydrogel that includes a plurality straight chain poly(ethylene glycol) macromers crosslinked with a plurality of n-arm-poly(ethylene glycol)macromers, (wherein n is 3 or more), by a plurality of β-hydroxythio-ether linkages, and
a plurality of cells encapsulated in the hydrogel, the hydrogel being cytocompatible, and, upon degradation, produce substantially non-toxic products.

13. The composition of claim 1, wherein the poly(ethylene glycol) macromers are linked to the n-arm-poly(ethylene glycol)macromers by reacting a plurality of diepoxy poly(ethylene glycol) macromers with a plurality n-arm-poly(ethylene glycol-thiol)macromers in a basic aqueous solution.

14. The composition of claim 12, further comprising at least one bioactive agent.

15. The composition of claim 14, further comprising at least one carrier material dispersed on or within the hydrogel, the carrier material including the bioactive agent and releasing the bioactive agent to modulate a function and/or characteristic of at least one of the cells.

16. The composition of claim 15, wherein the bioactive agent comprising at least one of DNA fragments, DNA plasmids, and/or interfering RNA molecules that modulates a function and/or characteristic of at least one of the cells.

17. The composition of claim 15, wherein the cells comprise progenitor cells, undifferentiated cells and/or differentiated cells.

18. A method of forming a hydrogel based biomaterial, the method comprising:
reacting a plurality of epoxy polymer macromers with a plurality thiolated polymer macromers in a basic aqueous solution to form a hydrogel, and
loading a plurality of cell and optionally, a bioactive agent, into the hydrogel during gelation of the hydrogel,
wherein the hydrogel is cytocompatible, and, upon degradation, produce substantially non-toxic products.

19. The method of claim 18, wherein the epoxy polymer macromers are epoxy poly(ethylene glycol) macromers and the thiolated polymer macromers are thiolated poly(ethylene glycol) macromers.

20. The method of claim 19, wherein the thiolated poly(ethylene glycol) macromers are n-arm-poly(ethylene glycol-thiol)macromers, wherein n is 3 or more, and the epoxy poly(ethylene glycol) macromers are diepoxy poly(ethylene glycol) macromers.

21. The composition of claim 18, wherein the bioactive agent comprising at least one of DNA fragments, DNA plasmids, and/or interfering RNA molecules that modulates a function and/or characteristic of at least one of the cells.
